(19) United States
(12) Patent Application Publication
(10) Pub. No.: US 2014/0120624 A1
(43) Pub. Date: May 1, 2014

(54) CASSETTES AND METHODS FOR TRANSFORMING AND SELECTING YEAST TRANSFORMANTS BY HOMOLOGOUS RECOMBINATION

(71) Applicants: Christele Perrin-East, Marseille (FR); Pablo Gluschankof, Marseille (FR)

(72) Inventors: Christele Perrin-East, Marseille (FR); Pablo Gluschankof, Marseille (FR)

(21) Appl. No.: 14/126,931

(22) PCT Filed: Jun. 20, 2011
(86) PCT No.: PCT/IB2011/002254

§ 371 Date: Jan. 16, 2014

(51) Int. Cl. C12N 15/81

(52) U.S. Cl. CPC C12N 15/81
USPC 435/477; 435/320.1; 435/256.8

(57) ABSTRACT

A method for selecting a transformed yeast cell having integrated a nucleic acid fragment of interest by homologous recombination, cassettes and kits for carrying out the method are described.

### FIELD OF THE INVENTION

The present invention relates to cassettes and methods for easily selecting the expected transformed yeast cells obtained by homologous recombination.

### BACKGROUND OF THE INVENTION

Yeast cells, and particularly Saccharomyces cerevisiae, are important organisms for the production of recombinant proteins as they meet both the demand for efficient mass production in case of compounds like technical enzymes and criteria of safety and authenticity in case of pharmaceutical proteins. Saccharomyces cerevisiae has been studied extensively, thus molecular and genetic tools are available for its manipulation and it is a successful industrial microorganism.

Different techniques are used to insert a DNA fragment of interest in a yeast cell through an expression vector. A first method is the well-known DNA sub-cloning through DNA modification enzymes (DNA restriction enzymes and DNA ligation enzymes). Briefly, In order to sub-clone a DNA fragment in a shuttle vector, a multi-cloning site (MCS) is generally present at the 3′ extremity of the promoter. This MCS DNA contains nucleotide sequences recognized by endonucleases (known as restriction enzymes) that are rarely found in genomes. After enzymatic digestion of both DNAs (the one to be sub-cloned, and the shuttle vector) by classical molecular procedures with the specific restriction enzymes present in both substrates (Sambrook J and Russel D. Molecular Cloning: A Laboratory Manual, 2001, 3rd edition, CSHL Press), ligation takes place and propagation of the plasmid is obtained in bacteria after bacteria transformation with the ligation product. A last step, in order to select the bacteria transformant carrying the expected plasmid, is performed by analysing the plasmid content (either by PCR or by restriction enzymes) of many individual transformants. Once the single bacteria transformant carrying the expected plasmid was selected, plasmid production, extraction and purification are performed using classical techniques. Several hundreds of nanograms of this purified plasmid are used to transform yeast.

A more simple method, for DNA subcloning in a shuttle yeast vector takes advantage of the homologous recombination event that takes place in yeast. In this case, there is no need to use bacteria as a host for plasmid propagation, as PCR amplified DNA fragments carrying at both sides sequences that are homologous to sequences located in the expression vector are used in a one step yeast co-transformation (single cut dephosphorylated vector and PCR amplified DNA).

However, homologous recombination does not permit to obtain 100% of expected transformants: yeast transformants obtained by this method carry either the empty vector (when homologous recombination did not take place) or the expected plasmid. The inventors show that about only 70% of total transformants harbours the expected plasmid. In order to select among the total yeast transformants the ones of interest, several steps consisting in yeast DNA purification and analysis or yeast functional assays have to be done on several single yeast transformant.

Thus, there is a need for new tools and methods for easily selecting yeast transformants of interest obtained by homologous recombination.

### SUMMARY OF THE INVENTION

The inventors developed new methods and cassette for easily selecting a transformed yeast cell having integrated a nucleic acid fragment of interest by homologous recombination in an expression vector. Said methods and cassette permit to save money and time: thus, they avoid the step of DNA purification for selecting expected yeasts and they allow selecting expected transformed yeast cells in one step.

The present invention relates to a method for selecting a transformed yeast cell having integrated a nucleic acid fragment of interest in a vector by homologous recombination, said method comprising the steps of:
(i) Contacting a yeast cell with:
   The vector comprising:
      a positive selection gene,
      a homologous recombination site comprising a 5′ and a 3′ recombination regions framing a restriction site, and
   A nucleic acid fragment of interest to insert by homologous recombination into the homologous recombination site of said vector, said nucleic acid fragment being flanked by regions substantially identical to the 5′ and 3′ recombination regions of the homologous recombination site, and
(ii) Transforming said yeast cell with said vector and said nucleic acid fragment of interest,
(iii) Selecting yeast cells harboring said vector with said positive selection gene,
   characterized in that:
A negative selection gene is further present in the vector downstream to the homologous recombination site and under the control of a promoter situated upstream to said homologous recombination site, said promoter and negative selection gene being operably linked in said vector before insertion of the DNA fragment of interest, and
(iv) The method further comprises a step of selecting yeast cells harboring the DNA fragment of interest using the negative selection gene.

The invention further provides a cassette comprising:
a homologous recombination site comprising a 5′ and a 3′ recombination regions framing a restriction site, and
a negative selection gene present downstream to said homologous recombination site,
   and a vector comprising:
an origin of replication,
a positive selection gene, said positive selection being under the control of a promoter,
a homologous recombination site comprising a 5′ and a 3′ recombination regions framing a restriction site, and
a negative selection gene present in the vector downstream to the homologous recombination site and under the control of a promoter situated upstream to said homologous recombination site, said promoter and negative selection gene being operably linked in said vector before insertion of the DNA fragment of interest and being under the control of a terminator.

The invention also relates to a method for obtaining a vector of the invention, said method comprising the step of integrating a cassette according to the invention into a vector, preferably a plasmid comprising:
a positive selection gene, and
a promoter,
   so as to place said negative selection gene, that is downstream to said homologous recombination site of the cassette, under the control of a said promoter present in the vector, said promoter and said negative selection gene being operably linked in the obtained vector.

The invention finally provides a kit comprising:
a cassette according to the invention, and
at least one yeast cell culture medium.

### BRIEF DESCRIPTION OF THE DRAWINGS

The drawings form part of the present specification and are included to further demonstrate certain aspects of the present invention. The invention may be better understood by reference to one or more of these drawings in combination with the detailed description of specific embodiments presented herein.

FIG. 1: create a cassette that could be inserted downstream the promoter region of pRS316 expression vector.

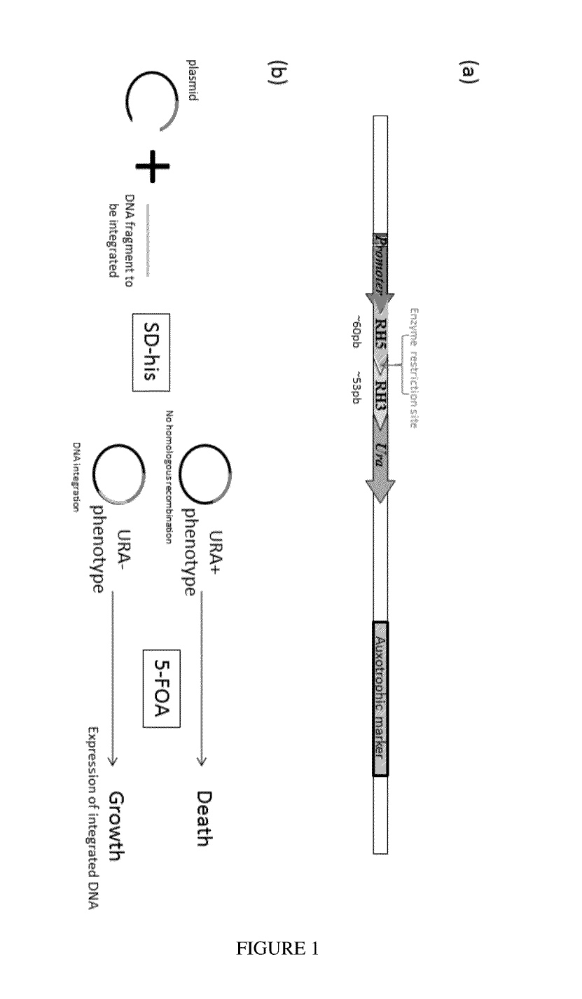

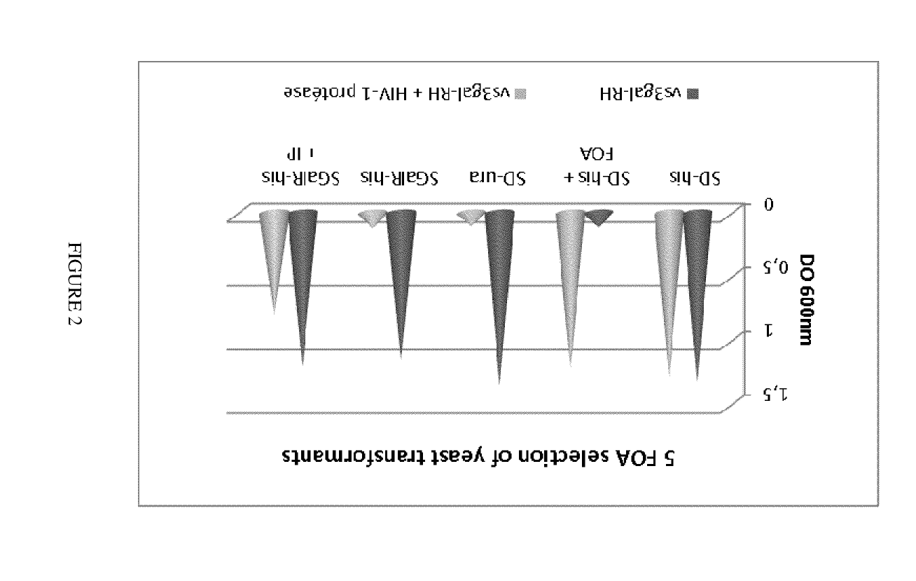

The rational of this construction is to create a cassette where the ura3 ORF will be located under the control of the promoter of a his3, Leu2 or Trp1 version of any shuttle yeast vector. The ura3 will be preceded by a 5′ homologous region (RH5), a unique restriction site and a 3′ homologous region (RH3) as shown in FIG. 1a.

The distance between the promoter and the start codon of the ura3 ORF, in this construction should lead to ura3 expression, whereas if the ura3 gene is located far away from the promoter, due to the insertion of a DNA fragment between RH5 and RH3 sequences, ura3 will not be expressed.

Incubation of transformants with 5-FOA will lead then to death of transformants expressing URA3, and growth of transformants harbouring the vector with a PCR amplified fragment inserted in it (FIG. 1b).

FIG. 2: Selection of yeast recombinants harbouring HIV-1 protease by 5-FOA incubation.

The vs3gal-RH is cut with XhoI, purified, mixed with PCR amplified sequence of HIV-1 protease presenting at its 5′ and 3′ ends the RH1-5′ and the RH1-3′ sequences and used to transform W303 yeast strain. Transformants grew at 30° C. for 24 hours in minimal media lacking histidine and 5-FOA, to a final concentration of 1 mg/ml, then left 48 hrs in minimal media lacking histidine. Cells were washed 3 times in sterile water and expression of HIV-1 Protease in galactose containing media was tested. Expression of HIV-1 Protease in yeast leads to cell death (Blanco et al, 2003, patent application WO 2011/007244). Results clearly demonstrate that the created DNA cassette is actually efficient for selecting, through 5-FOA incubation, only clones where the DNA fragment is inserted into the vector as shown by the arrest of cell growth in galactose (SGalR-his media), prevented by addition of a specific inhibitor of the HIV-1 protease (IP).

### DETAILED DESCRIPTION OF THE INVENTION

### Methods of the Invention

A first object of the invention relates to a method for selecting a transformed yeast cell having integrated a nucleic acid fragment of interest in a vector by homologous recombination, said method comprising the steps of:
(i) Contacting a yeast cell with:
   The vector comprising:
      a positive selection gene,
      a homologous recombination site comprising a 5′ and a 3′ recombination regions framing a restriction site, and
   A nucleic acid fragment of interest to insert by homologous recombination into the homologous recombination site of said vector, said nucleic acid fragment being flanked by regions substantially identical to the 5′ and 3′ recombination regions of the homologous recombination site, and
(ii) Transforming said yeast cell with said vector and said nucleic acid fragment of interest,
(iii) Selecting yeast cells harboring said vector with said positive selection gene,
   Characterized in that:
A negative selection gene is further present in the vector downstream to the homologous recombination site and under the control of a promoter situated upstream to said homologous recombination site, said promoter and negative selection gene being operably linked in said vector before insertion of the DNA fragment of interest, and
(iv) The method further comprises a step of selecting yeast cells harboring the DNA fragment of interest using the negative selection gene.
   It should be noticed that with the method of the invention:
The transformed yeast cells which did not integrate the DNA fragment of interest in the vector die at the step (iv) because of the expression of the negative selection gene which is still operably linked with said promoter, and
The transformed yeast cells having integrated the DNA fragment of interest in the vector live after the step (iv) because of the absence of expression of the negative selection gene which is no more operably linked with its promoter when the nucleic acid fragment of interest is inserted between said negative selection gene and said promoter.

The term “transformation” or “transforming” refers to the transfer of a DNA fragment, a plasmid or a vector into a host organism. Host organisms containing such DNA fragment, plasmid or vector are called “recombinant” or “transformed” organisms.

In the method of the invention, the transformed organism is a yeast, such as Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferably, the transformed yeast cell is Saccharomyces cerevisiae.

The terms “vector” or “plasmid” refer to extra chromosomal elements often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

Vectors and plasmids of the invention thus comprise an origin of replication functional in yeast. The term “origin of replication functional in yeast” refers to any nucleic acid sequence which allows replication of a vector or a plasmid independently from the chromosome. Generally, the origin of replication is functional in at least one or more of the following: Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Suitable origins of replication include, for example, ars 1, centromere ori, and 2μ ori.

In one embodiment, the method of the invention comprises a previous step of obtaining the vector of the invention by integration of a cassette comprising:
a homologous recombination site comprising a 5′ and a 3′ recombination regions framing a restriction site, and
a negative selection gene downstream to said homologous recombination site,
   into a vector, preferably a plasmid comprising:
a positive selection gene, and
a promoter,
   so as to place said negative selection gene, that is downstream to said homologous recombination site of the cassette, under the control of a said promoter present in the vector, upstream to said homologous recombination site of the cassette, said promoter and said negative selection gene being operably linked in the obtained vector.

According to the invention, the term “cassette” should be considered as an element comprising specific nucleic acid sequences to integrate by any means, for example by enzyme digestion and DNA ligation, by recombination, more particularly by homologous recombination into a plasmid.

As used herein, the term “promoter” refers to a DNA sequence capable of directing transcription (thus expression) of a gene or a coding sequence which is operably linked to said promoter.

Generally, the term “Operably linked” means that the transcriptional regulatory nucleic acid is positioned relative to any coding sequence in such a manner that transcription is initiated. As used herein, this will mean that the promoter is positioned 5′ to the coding region, at a distance allowing the expression of said coding region. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions.

There are different kinds of promoters. A promoter as used herein may be an inducible or regulated promoter (its activity is up or down regulated by the presence or absence of biotic or abiotic factors) or a constitutive promoter (it leads to a gene expression in most cell types at most times). A promoter used according to the invention may be a strong promoter (leading to a high gene expression) or weak promoter (leading to a low gene expression) (Maya, D, Quintero, M J, Munoz-Centeno, M, Chavez, S, Systems for applied gene control in Saccharomyces cerevisiae. 2008. Biotechnol Lett 30:979-987).

Promoters used according to the invention are functional in yeast cells. Examples of promoters functional in yeast that could be used in the present invention comprise, but are not limited to: the GAL1 promoter (SEQ ID n°1), the ADH1 promoter (SEQ ID n°2) and the strong GPD (Glyceraldehyde 3P DH) promoter (SEQ ID n°3). (MAYA et al., Biotechnol. Lett., vol. 30, p:979-987, 2008).

By “selection gene” (or “reporter gene”, “selectable gene”) is meant a gene that by its presence in a host cell, i.e. upon expression, can allow the host to be distinguished from a cell that does not contain the selectable gene. Selectable genes can be classified into several different types, including positive and negative selection genes. It may be the nucleic acid or the protein expression product that causes a particular effect in certain conditions. Additional components, such as substrates, ligands, etc. may be additionally added to allow selection or sorting on the basis of the selectable gene.

As used herein, selection genes of the invention begin by a start codon and terminate by a stop codon, and are preceded by a promoter (that allows and control gene expression), and followed by a terminator to be functional.

A “positive selection gene” is a nucleic acid sequence that allows the survival of cells expressing the positive selection gene under growth conditions that kill or prevent growth of cells lacking said gene. An example of a positive selection gene is a nucleic acid sequence which promotes expression of an antibiotic resistance gene such as neomycin resistance gene or kanamycin resistance gene. Cells not containing the neomycin resistance gene are selected against by application of G418, whereas cells expressing the neomycin resistance gene are not harmed by G418 (positive selection).

Preferred positive selection genes functional in yeast are survival genes which include ADE2, HIS3, LEU2, or TRP1. ALG7 confers increased resistance to tunicamycin, the neomycin phosphotransferase gene confers resistance to G418, and the CUP1 gene, which allows yeast to grow in the presence of copper ions.

According to the invention, the positive selection gene present in the vector is a classical positive gene selection functional in yeast placed under the control of a functional yeast promoter.

Preferably, said positive selection gene is the HIS3 gene (SEQ ID n°4). For selecting yeast cell, lacking a functional HIS3 gene in its genome, harboring the vector according to step (iii) of the method of the invention, cultured yeast cells are placed on a medium lacking histidine. Only the yeast cells expressing the His3 gene are able to grow in this medium.

A “negative selection gene” is a nucleic acid sequence that kills, prevents growth of or otherwise selects against cells expressing said negative selection gene, usually upon application of an appropriate exogenous agent. An example of a negative selection gene is a nucleic acid sequence which promotes expression of the thymidine kinase gene of herpes simplex virus (HSV-TK). Cells expressing HSV-TK are selected against by application of ganciclovir (negative selection), whereas cells not expressing the gene are relatively unharmed by ganciclovir. The terms are further defined, and methods further explained, by U.S. Pat. No. 5,464,764, which is herein incorporated by reference. Another example of a negative selection gene is the “URA” (orotidine 5′ phosphate decarboxylase) gene. “URA3” is the “URA” gene of the budding yeast S. cerevisiae and K. lactis, while “URA4” is the “URA” gene of S. pombe. As used herein, the term “URA3 gene” refers to a gene encoding an enzyme involved in the synthesis of pyrimidine ribonucleotides and necessary for cell growth in a medium lacking uracil or uridine. Said enzyme also converts the 5-Fluoroorotic acid (5-FOA) into a toxic compound 5-fluorouracil causing cell death. The URA3 gene can be used as a positive and negative selection gene for DNA transformations, particularly yeast DNA transformations.

A “URA3 gene” as used herein includes a URA3 gene derived from any yeast, preferably from Saccharomyces cerevisiae or Kluyveromyces lactis, as well as such URA3 function-conservative variants harboring mutations and keeping the URA3 activities cited above. An example of sequences of Saccharomyces cerevisiae URA3 gene is given in SEQ ID n°5.

As used herein, a “variant” or a “function-conservative variant” includes a nucleic acid sequence in which one or several nucleotides have been changed and which has at least 80% nucleotide identity as determined by BLAST or FASTA algorithms, preferably at least 90%, most preferably at least 95%, and even more preferably at least 99%, and which has the same or substantially similar properties or functions as the native or parent gene to which it is compared.

Preferably, said negative selection gene is the URA3 gene. For selecting yeast cell harboring the nucleic acid fragment of interest according to step (iv) of the method of the invention, yeast cells are placed on a medium containing 5-FOA.

In the cells that do not harbor said fragment, the URA3 gene and the promoter which controls URA3 gene expression in the vector of the invention are operably linked, and URA3 is expressed. 5-FOA being converted into a toxic compound when URA3 gene is expressed, yeast cells that do not harbor said fragment die.

In the cells that harbor said fragment, the URA3 gene and said promoter are separated by said fragment, and no more operably linked according to the invention. Thus, the URA3 gene is not expressed and 5-FOA is not toxic for these cells which are still living and continue to grow.

Indeed, according to the invention, the negative selection gene is placed under the control of a promoter functional in yeast. In the vector of the invention, the negative selection gene is separated from said promoter by the homologous recombination site, but is operably linked to said promoter (e.g. is near enough to said promoter to allow said promoter to control the expression of the negative selection gene).

When homologous recombination has occurred, the nucleic acid fragment of interest is inserted in the homologous recombination site, thus between the promoter and the negative selection gene. In that case, the promoter and the negative selection gene are not operably linked (because of a too important distance between them).

The inventors showed that the notion of “operably linked” depend on the kind of promoters.

In a particular embodiment, the invention relates to said method for selecting a transformed yeast cell having integrated a nucleic acid fragment of interest by homologous recombination wherein:
the negative selection gene is under the control of a strong promoter, preferably the GPD promoter, and
the insertion of the nucleic acid fragment of interest of the invention by homologous recombination separates the negative selection gene from its promoter by at least 2000 pb, particularly at least 3000 pb, more particularly at least 4000 pb, preferably at least 5000 pb.

In a particular embodiment, the invention relates to said method for selecting a transformed yeast cell having integrated a nucleic acid fragment of interest in a vector by homologous recombination wherein:
the negative selection gene is under the control of an inducible promoter (which is leaky in a non inducible medium), preferably the GAL-1 promoter, and
the insertion of the nucleic acid fragment of interest of the invention by homologous recombination separates the negative selection gene from its promoter by at least 120 pb, particularly at least 150 pb, more particularly at least 188 pb.

In a particular embodiment, the invention relates to said method for selecting a transformed yeast cell having integrated a nucleic acid fragment of interest by homologous recombination wherein:
the negative selection gene is under the control of an intermediate promoter, preferably the ADH1 promoter, and
the insertion of the nucleic acid fragment of interest of the invention by homologous recombination separates the negative selection gene from its promoter by at least 120 pb, particularly at least 150 pb, more particularly at least 169 pb.

In one embodiment of the invention, both steps of selection (iii) and (iv) may be realized simultaneously or separately, preferably simultaneously.

The term “homologous recombination site” refers to a site that allows the introduction of the nucleic acid fragment of interest into a vector or a shuttle vector by homologous recombination. Homologous recombination is, briefly, the process of strand exchange that can occur spontaneously with the alignment of homologous sequences (i.e. sets of complementary strands). As is known in the art, yeasts are efficient at homologous recombination. Orr-Weaver, et al, Proc. Natl. Acad. Sci. USA 78: 6345-6358. 1981; Ma, et al., Gene, 58:201-216 (1987); Petermann, Nucleic Acids Res., 26(9):2252-2253 (1998); each incorporated herein by reference. Methods and conditions allowing homologous recombination are well known in the art. Thus, in general, the homologous recombination site contains two distinct, but generally contiguous, regions. The first region, referred to herein as the 5′ region, is generally identical to the 5′ region flanking the nucleic acid fragment of interest to insert into the vector. The second region, referred to herein as the 3′ region, is generally identical to the 3′ region flanking the nucleic acid fragment of interest to insert into the vector. Preferably, the 5′ and 3′ regions are each at least 12 or 15 nucleic acids long. More preferably, the 5′ and 3′ regions are each at least about 20 or 30 nucleic acids long, and more preferably at least about 50 nucleic acids long, and most preferably about 60 nucleic acids long. According to the invention, the homologous recombination site sequence refers to any nucleic acid sequence which is unique to the vector in that the vector does not comprise another sequence corresponding to the sequence of the homologous recombination site and which is homologous which the flanking regions of the nucleic acid fragment of interest to insert.

Examples of 5′ and 3′ homologous recombination regions pairs are provided in the examples (SEQ ID n°6 and SEQ ID n°7 respectively or SEQ ID n°16 and SEQ ID n°17 respectively).

The term “nucleic acid fragment of interest” as used herein refers to any nucleic acid to insert into a vector at a homologous recombination site.

According to the invention, the nucleic acid fragment of interest is flanked by 5′ and 3′ regions identical (or substantially identical) to the 5′ and 3′ regions of a homologous recombination site on the vector provided herein. Thus, when the nucleic acid fragment of interest is inserted into the vector, the 5′ and 3′ regions flanking the nucleic acid fragment of interest replace the 5′ and 3′ regions of the homologous recombination site during homologous recombination.

As used herein, substantial identity would be about 80%, particularly 90%, preferably 95%, more preferably 99% of identity between the bases of the DNA fragment to insert and the recombination regions.

According to the invention, said nucleic acid fragment of interest may be any DNA sequence coding (or not) for a protein to produce. For example, said fragment may be genes coding for any foreign or yeast protein, or non-coding nucleic acid sequences Preferably, said fragment is selected in the group comprising: a HIV-1 or HIV-2 reverse transcriptase gene sequence, a HIV-1 or HIV-2 protease gene sequence, a HIV-1 or HIV-2 integrase gene sequence, a HIV-1 or HIV-2 gag-pol precursor gene entire or partial sequence.

In one embodiment of the invention, the method for selecting a transformed yeast cell having integrated a nucleic acid fragment of interest by homologous recombination, said method comprises a further previous step of synthesizing a nucleic acid fragment of interest, said fragment comprising the sequence of the nucleic acid to study or to produce flanked by sequences substantially identical to the 5′ and 3′ regions of a homologous recombination site on the vector of step (i).

As used herein, the term “restriction site”, or “restriction recognition site”, is a location on a DNA molecule containing specific sequences of nucleotides which are recognized by restriction enzymes. A particular restriction enzyme may cut the sequence between two nucleotides within its recognition site, or somewhere nearby. According to the invention, the restriction site is unique into the vector of invention, in that the vector does not comprise another sequence corresponding to said restriction site. Any restriction site may be used; examples of restriction sites comprise, but are not limited to: NotI, BamHI, XhoI, EcoRI, NcoI, SacI, SalI, SmaI, PvuII, ScaI . . . . The restriction site of the invention allows linearizing the vector of the invention, for homologous recombination. Indeed, the homologous recombination is done with a single cut dephosphorylated vector.

### Cassettes and Vectors of the Invention

A second object of the invention relates to a cassette comprising:
a homologous recombination site comprising a 5′ and a 3′ recombination regions framing a restriction site, and
a negative selection gene present downstream to said homologous recombination site.

According to the invention, said cassette is used to be inserted into a vector, preferably a plasmid, comprising a positive gene selection and a promoter, so as to place the negative selection gene of the cassette (that is downstream to said homologous recombination site of said cassette) under the control of the promoter of the vector, said promoter and said negative selection gene being operably linked in the obtained vector.

The obtained vector (e.g. the plasmid in which said cassette has been correctly inserted) is used for carrying out the method of the invention.

According to the invention, the vector comprises an origin of replication and the positive selection gene is under the control of a promoter. Each of the selection genes of the invention is also under the control of a terminator.

As used herein, the term “terminator”, or “transcription terminator” is a region of nucleic acid sequence that marks the end of a gene or operon on genomic DNA for transcription. Terminators being functional in yeast cells are well known in the art, like ADH1 terminator, STE2 terminator, CycE1.

A third object of the invention relates to a vector comprising:
an origin of replication,
a positive selection gene, said positive selection being under the control of a promoter,
a homologous recombination site comprising a 5′ and a 3′ recombination regions framing a restriction site, and
a negative selection gene present in the vector downstream to the homologous recombination site and under the control of a promoter situated upstream to said homologous recombination site, said vector before insertion of the DNA fragment of interest and being under the control of a terminator.

According to the invention, the positive selection gene and the negative selection gene are each under the control of a promoter and a terminator.

In one embodiment of the invention, said vector is a shuttle vector, e.g. a vector constructed so that it can propagate in both yeast and bacteria cells (for examples Escherichia coli).

Thus, in a particular embodiment of the invention, said vector further comprises a bacterial origin of replication and a bacterial positive gene selection (for example the ampicillin resistance gene).

According to the invention, said vector may be used to transform a yeast cell in order to insert into said cell a nucleic acid fragment of interest by homologous recombination and to select transformed yeast cells that actually harbor said nucleic acid fragment of interest in the expression vector, by the selection method described above.

In one embodiment, the invention relates to a method for obtaining a vector of the invention, said method comprising integrating a cassette comprising:
a homologous recombination site comprising a 5′ and a 3′ recombination regions framing a restriction site, and
a negative selection gene downstream to said homologous recombination site,
   into a vector comprising:
a positive selection gene, and
a promoter,

so as to place said negative selection gene, that is downstream to said homologous recombination site of the cassette, under the control of a said promoter present in the vector, said promoter and said negative selection gene being operably linked in the obtained vector.

The cassette and the vector of the invention are described in more details above.

### Kits of the Invention

A fourth object of the invention relates to a kit for carrying out the method of the invention.

In one embodiment, the invention relates to a kit comprising:
a cassette of the invention,
at least one yeast cell culture medium.

According to the invention, said kit is used to insert said cassette into a vector and to transform a yeast cell with said vector and a nucleic acid fragment of interest for carrying out the method of the invention.

Said vector may be any functional vector comprising a positive selection gene and a promoter.

In a particular embodiment, said kit may further comprise:
Selective medium or compounds allowing the selection by the negative and/or positive selection gene(s) present in the vector of the invention,

In another particular embodiment, said kit may further comprise:
Nucleic fragments sequences identical or substantially identical to the 5′ and 3′ homologous recombination regions.

In one embodiment, the invention relates to a kit comprising:
a vector according to the invention as defined above, and
at least one culture yeast cell culture medium.

In a particular embodiment, said kit may further comprise:
Selective medium or compounds allowing the selection by the negative and/or positive selection gene(s) present in the vector of the invention,

In another particular embodiment, said kit may further comprise:
Nucleic fragments sequences identical or substantially identical to the 5′ and 3′ homologous recombination regions.

In another embodiment, the kit of the invention further comprises a yeast cell, preferably a Saccharomyces cerevisiae yeast cell.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

The present kit can also include one or more reagents, buffers, hybridization media, nucleic acids, primers, nucleotides, probes, molecular weight markers, enzymes, solid supports, databases, computer programs for calculating dispensation orders and/or disposable lab equipment, such as multi-well plates, in order to readily facilitate implementation of the present methods. Enzymes that can be included in the present kit include nucleotide polymerases and the like. Solid supports can include beads and the like whereas molecular weight markers can include conjugable markers, for example biotin and streptavidin or the like.

In one embodiment, the kit is made up of instructions for carrying out the method described herein. The instructions can be provided in any intelligible form through a tangible medium, such as printed on paper, computer readable media, or the like.

### EXAMPLES

The following examples describe some of the preferred modes of making and practicing the present invention. However, it should be understood that the examples are for illustrative purposes only and are not meant to limit the scope of the invention.

### Example 1

### Insertion of a “Suicidal Cassette” Downstream the GAL Promoter Region of a Modified pRS Expression Vector

The coding region for URA3 amplified by PCR using primers 5SacUra (SEQ ID n°8) and 3UraSac (SEQ ID n°9) is digested by Sad for 1 hr at 37° C. to be cloned under the control of the GAL1 promoter into the modified version of the pRS vector also digested by SacI. The newly created cassette, situated downstream the GAL1 promoter, contains the following sequences:
RH15′ (SEQ ID n°6)
a unique XhoI restriction enzyme site
RH13′ (SEQ ID n°7)
the URA3 ORF.

In this construction the start codon of the URA3 gene is located 88 bp downstream the end of the GAL1 promoter. In this newly created expression vector, vs3gal-RH, the distance between the GAL1 promoter and the start codon of the URA3 ORF leads, due to the “leaky” character of this promoter in the HIS3 version of the pRS backbone, to URA3 expression even in glucose media (tablet).

TABLE 1
Growth of yeast transformed with vector vs3gal-RH carrying the
“suicidal cassette” in GALL inducible and non-inducible media.

| | Growth in SD- | Growth in | Growth in SD- |
|---|---|---|---|
| | his | SGalR-ura | ura |
| | (Absorbance at | (Absorbance at | (Absorbance at |
| | 600 nm) | 600 nm) | 600 nm) |
| vs3gal-RH | 1.33 | 1.29 | 1.36 |

This newly produced vector was single cut with XhoI, purified, mixed with PCR amplified sequence of HIV-1 protease presenting at its 5′ and 3′ ends the RH1-5′ and the RH1-3′ sequences and used to transform W303 yeast strain.

Transformants grew at 30° C. for 24 hours in minimal media lacking histidine and 5-FOA to a final concentration of 1 mg/ml then left 48 hrs in minimal media lacking histidine. Cells were washed 3 times in sterile water and expression of HIV-1 Protease in galactose containing media was tested.

It was previously reported that expression of HIV-1 Protease in S. cerevisiae induces cell death. FIG. 2 shows cell growth of W303 transformed with linearized vs3gal-RH vector with or without the PCR amplified viral protease gene. Analysis of the shown results clearly demonstrate that the created DNA cassette is actually efficient for selecting, through 5-FOA incubation, only clones where the DNA fragment is inserted into the vector (FIG. 2).

Experiments done on vs3gal-RH vector have shown that the “suicide character” of the created cassette is effective when the URA3 start codon is located at a distance smaller than about 188 bp downstream the end of the GAL1 Promoter (88 bp length in the absence of any DNA fragment plus the presence of an inserted fragment of 100 bp) (table2).

TABLE 2
100 bp length DNA fragment insertion suppress 5-FOA lethality
of vs3gal-RH vector.

| | Growth in SD- | Growth in SD- | Growth in SD- |
|---|---|---|---|
| | his | his + 5-FOA | ura |
| | (Absorbance at | (Absorbance at | (Absorbance at |
| | 600 nm) | 600 nm) | 600 nm) |
| vs3gal-RH | 1.35 | 0.09 | 1.38 |
| vs3gal-RH + HIV-1 | 1.7 | 1.055 | 0.12 |
| protéase | | | |
| vs3gal-RH + 200 pb | 0.99 | 0.505 | 0.09 |
| DNA fragments | | | |
| vs3gal-RH + 100 pb | 1.32 | 1.27 | 0.1 |
| DNA fragment | | | |

W303 yeast strain was transformed with the linearized and dephosphorylated vs3gal-RH vector and a DNA fragment flanked by regions identical to the 5′ and 3′ recombination regions of the homologous recombination site of the vector. The obtained transformants were tested for growth in different selective media in the presence or absence of 5-FOA for 2 days. Plasmids harboring DNA fragments of at least 100pb length conferred 5-FOA resistance.

### Example 2

### Insertion of a “Suicidal Cassette” Downstream the ADH1 Promoter Region of p415ADH1 Expression Vector

p415ADH vector (Mumberg, D., Müller, R, Funk, M. 1995. Gene. 156:119-122. Yeast vectors for the controlled expression of heterologous proteins in different genetic backgrounds) was cut with restriction enzyme XhoI located in its multi-cloning site, and the ORF region of the URA3 gene was inserted at that position. The cassette, object of the invention, encompasses the following sequences:
the 5′ homologous recombination region RH5p4xx (SEQ ID n°16),
a unique restriction enzyme site,
the 3′ homologous recombination region RH3p4xx (SEQ ID n°17),
the URA3 ORF.

In this cassette the start codon of the URA3 gene is located 69 bp downstream of the end of the ADH1 promoter. When URA-yeast cells were transformed with this vector (vs5ADH-RH), the URA3 gene is expressed, as the transformants grew on minimal synthetic media lacking uracil.

We tested the “suicide character” of the resulted expression vector by sub-cloning the ORF region of TRP1 gene, of about 670 bp, downstream the ADH1 promoter and upstream the URA3 gene as follows:

The coding region for the auxotrophic marker TRP1 was amplified by PCR using primers (SEQ ID n°10 and SEQ ID n°11).

The fragment carrying the new selectable marker was then incorporated into the vs5ADH-RH vector (linearized by the restriction enzyme HindIII) by homologous recombination in CB018 or W303 yeast strain.

When URA-, TRP-yeast cells were transformed with this plasmid, URA3 gene was not expressed, as none of the transformants grew in minimal synthetic media lacking uracil, but TRP1 gene was expressed, as the transformants grew on minimal synthetic media lacking tryptophan.

Yeast transformants carrying the new plasmid expressed TRP1 but not URA3 gene, implying that a 675 bp distance from the 3′ end of the ADH1 promoter does not allow URA3 gene to be expressed (table3).

Experiments done on vs5ADH-RH vector have shown that the “suicide character” of the created new cassette is effective when the URA3 start codon is located at a distance smaller than about 169 bp downstream the end of the ADH1 Promoter (table3).

TABLE 3
100 bp length DNA fragment insertion suppress 5-FOA lethality of vs5ADH-RH
vector.

| | | Growth in SD- | Growth in SD- | Growth in SD- |
|---|---|---|---|---|
| | Growth in SD | leu + 5-FOA | ura | trp |
| | (Absorbance at | (Absorbance at | (Absorbance at | (Absorbance at |
| | 600 nm) | 600 nm) | 600 nm) | 600 nm) |
| vs5ADH-RH | 1.27 | 0.09 | 1.31 | 0.09 |
| vs5ADH-RH + trp1 | 1.14 | 0.92 | 0.08 | 1.04 |
| vs5ADH-RH + 300 pb | 1.14 | 0.93 | 0.1 | 0.09 |
| DNA fragment | | | | |
| vs5ADH-RH + 100 pb | 1.22 | 0.95 | 0.08 | 0.1 |
| DNA fragment | | | | |

W303 yeast strain was transformed with the linearized and dephosphorylated vs5ADH-RH vector and a DNA fragment flanked by regions identical to the 5′ and 3′ recombination regions of the homologous recombination site of the vector The DNA fragment was the full length ORF of the TRP1 gene or a smaller fragment of the said ORF. The obtained transformants were tested for growth in different selective media in the presence or absence of 5-FOA for 2 days. Plasmids harboring DNA fragments of at least 100pb length conferred 5-FOA resistance.

### Example 3

### Insertion of the “Suicidal Cassette” Created in Example 2 Downstream the GPD Promoter Region of p424GPD Expression Vector

The “suicidal cassette” created in example 2 was excised from vs5ADH-RH vector using restriction enzymes Sad and KpnI. The purified SacI-KpnI cassette was inserted into p424GPD vector (Mumberg, D., Midler, R, Funk, M. 1995. Gene. 156:119-122. Yeast vectors for the controlled expression of heterologous proteins in different genetic backgrounds) previously cut with the same enzymes resulting in vs4GPD-RH vector.

When URA-yeast cells were transformed with this vector (vs4GPD-RH), the URA3 gene is expressed, as the transformants grew on minimal synthetic media lacking uracil.

We tested the “suicide character” of the resulted expression vector by sub-cloning the ORF region of HIS3 gene, of about 660 bp, downstream the GPD promoter and upstream the URA3 gene as follows:

The coding region for the auxotrophic marker HIS3 was amplified by PCR using primers (SEQ ID n°12 and SEQ ID n°13).

The fragment carrying the new selectable marker was then incorporated into the vs4GPD-RH vector (linearized by the restriction enzyme EcoRI) by homologous recombination in W303 yeast strain.

When URA-, HIS-yeast cells were transformed with this plasmid, URA3 and HIS3 genes are expressed, as the transformants grew on minimal synthetic media lacking uracil and histidine.

We then tested the “suicide character” of the resulted expression vector by sub-cloning the ORF region of LEU2 gene, of about 1 095 bp, downstream the GPD promoter and upstream the URA3 gene as follows:

The coding region for the auxotrophic marker LEU2 was amplified by PCR using primers (SEQ ID n°14 and SEQ ID n°15).

The fragment carrying the new selectable marker was then incorporated into the vs4GPD-RH vector (linearized by the restriction enzyme EcoRI) by homologous recombination in CB018 or W303 yeast strain.

When URA-, LEU-yeast cells were transformed with this plasmid, URA3 and LEU2 genes are expressed, as the transformants grew on minimal synthetic media lacking uracil and leucine.

1. A method for selecting a transformed yeast cell having integrated a nucleic acid fragment of interest in a vector by homologous recombination, said method comprising the steps of:
(i) Contacting a yeast cell with:
The vector comprising:
a positive selection gene,
a homologous recombination site comprising a 5′ and a 3′ recombination regions framing a restriction site, and
A nucleic acid fragment of interest to insert by homologous recombination into the homologous recombination site of said vector, said nucleic acid fragment being flanked by regions substantially identical to the 5′ and 3′ recombination regions of the homologous recombination site, and
(ii) Transforming said yeast cell with said vector and said nucleic acid fragment of interest,
(iii) Selecting yeast cells harboring said vector with said positive selection gene,
Characterized in that:
A negative selection gene is further present in the vector downstream to the homologous recombination site and under the control of a promoter situated upstream to said homologous recombination site, said promoter and negative selection gene being operably linked in said vector before insertion of the DNA fragment of interest, and
(iv) The method further comprises a step of selecting yeast cells harboring the DNA fragment of interest using the negative selection gene.

2. The method according to claim 1, said method further comprising a previous step of obtaining the vector of the invention by integration of a cassette comprising:
a homologous recombination site comprising a 5′ and a 3′ recombination regions framing a restriction site, and
a negative selection gene downstream to said homologous recombination site,
into a vector comprising:
a positive selection gene, and
a promoter,
so as to place said negative selection gene, that is downstream to said homologous recombination site of the cassette, under the control of a said promoter present in the vector, said promoter and said negative selection gene being operably linked in the obtained vector.

3. The method according to claim 1, said method comprising a further previous step of synthesizing a nucleic acid fragment of interest, said fragment comprising the sequence of the nucleic acid to study or to produce flanked by sequences substantially identical to the 5′ and 3′ regions of a homologous recombination site on the vector of step (i).

4. The method according to claim 1, wherein said negative selection gene is the URA3 gene.

5. The method according to claim 4, wherein said promoter controlling the negative selection gene expression is the GAL1.

6. The method according to claim 5, wherein:
the negative selection gene URA3 is under the control of an inducible promoter (which is leaky in a non inducible medium), preferably the GAL-1 promoter, and
the insertion of the nucleic acid fragment of interest of the invention by homologous recombination separates the negative selection gene from its promoter by at least 120 base pairs, most preferably by at least 188 base pairs.

7. The method according to claim 4, wherein said promoter controlling the negative selection gene expression is the ADH1.

8. The method according to claim 7, wherein:
the negative selection gene URA3 is under the control of the ADH-1 promoter, and
the insertion of the nucleic acid fragment of interest of the invention by homologous recombination separates the negative selection gene from its promoter by at least 100 base pairs, most preferably by at least 169 base pairs.

9. The method according to claim 1, wherein selection steps (iii) and (iv) are realized simultaneously.

10. A cassette comprising:
a homologous recombination site comprising a 5′ and a 3′ recombination regions framing a restriction site, and
a negative selection gene present downstream to said homologous recombination site.

11. The cassette of claim 10 wherein said negative selection gene is the URA3 gene.

12. A vector comprising:
an origin of replication,
a positive selection gene, said positive selection being under the control of a promoter,
a homologous recombination site comprising a 5′ and a 3′ recombination regions framing a restriction site, and
a negative selection gene present in the vector downstream to the homologous recombination site and under the control of a promoter situated upstream to said homologous recombination site, said promoter and negative selection gene being operably linked in said vector before insertion of the DNA fragment of interest and being under the control of a terminator.

13. A method for obtaining a vector comprising: an origin of replication, a positive selection gene, said positive selection being under the control of a promoter, a homologous recombination site comprising a 5′ and a 3′ recombination regions framing a restriction site, and a negative selection gene present in the vector downstream to the homologous recombination site and under the control of a promoter situated upstream to said homologous recombination site, said promoter and negative selection gene being operably linked in said vector before insertion of the DNA fragment of interest and being under the control of a terminator, said method comprising integrating a cassette according to claim 10 into a vector comprising:
a positive selection gene, and
a promoter,
so as to place said negative selection gene, that is downstream to said homologous recombination site of the cassette, under the control of a said promoter present in the vector, said promoter and said negative selection gene being operably linked in the obtained vector.

14. A kit comprising:
a cassette according to claim 10,
at least one yeast cell culture medium.

15. The kit according to claim 14, said kit further comprising:
Selective media or compounds allowing the selection by the negative and/or positive selection gene(s) present in the vector of the invention, and/or
Nucleic fragments sequences identical or substantially identical to the 5′ and 3′ homologous recombination regions.

16. The method according to claim 2, said method comprising a further previous step of synthesizing a nucleic acid fragment of interest, said fragment comprising the sequence of the nucleic acid to study or to produce flanked by sequences substantially identical to the 5′ and 3′ regions of a homologous recombination site on the vector of step (i).

17. The method according to claim 1, wherein said promoter controlling the negative selection gene expression is the GAL1.

18. The method according to claim 1, wherein said promoter controlling the negative selection gene expression is the ADH1.


SEQUENCE LISTING (17 sequences)

SEQ ID NO: 1 - DNA, 530 bp, Saccharomyces cerevisiae
ccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaattaag

SEQ ID NO: 2 - DNA, 690 bp, Saccharomyces cerevisiae
tgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactatctc

SEQ ID NO: 3 - DNA, 655 bp, Saccharomyces cerevisiae
agtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggat

SEQ ID NO: 4 - DNA, 663 bp, Saccharomyces cerevisiae
atgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtag

SEQ ID NO: 5 - DNA, 804 bp, Saccharomyces cerevisiae
atgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 6 - DNA, 39 bp, Artificial Sequence
  homologous recombination region
tatactttaacgtcaaggagaaaaaaccccggatccatg

SEQ ID NO: 7 - DNA, 42 bp, Artificial sequence
  homologous recombination region
taagagctcatgtcgaaagctacatataaggaacgtgctgct

SEQ ID NO: 8 - DNA, 22 bp, Artificial sequence
  primer
ttagcgagctcatgtcgaaagc

SEQ ID NO: 9 - DNA, 23 bp, Artificial sequence
  primer
ccagcaaaactaagagctccact

SEQ ID NO: 10 - DNA, 54 bp, Artificial sequence
  primer
tctagaactagtggatcccccgggctgcaggaattcatgtctgttattaatttc

SEQ ID NO: 11 - DNA, 58 bp, Artificial Sequence
  primer
agcagcacgttccttatatgtagctttcgacatctcgagctatttcttagcatttttg

SEQ ID NO: 12 - DNA, 53 bp, Artificial Sequence
  primer
tctagaactagtggatcccccgggctgcaggaattcatgacagagcagaaagc

SEQ ID NO: 13 - DNA, 55 bp, Artificial Sequence
  primer
agcagcacgttccttatatgtagctttcgacatctcgagctacataagaacacct

SEQ ID NO: 14 - DNA, 52 bp, Artificial Sequence
  primer
tctagaactagtggatcccccgggctgcaggaattcatgtctgcccctaaga

SEQ ID NO: 15 - DNA, 55 bp, Artificial Sequence
  primer
agcagcacgttccttatatgtagctttcgacatctcgagttaagcaaggattttc

SEQ ID NO: 16 - DNA, 39 bp, Artificial Sequence
  homologous recombination region
tctagaactagtggatcccccgggctgcaggaattcatg

SEQ ID NO: 17 - DNA, 42 bp, Artificial Sequence
  homologous recombination region
taactcgagatgtcgaaagctacatataaggaacgtgctgct